(19) United States
(12) Patent Application Publication
(10) Pub. No.: US 2017/0315119 A1
(43) Pub. Date: Nov. 2, 2017

(54) URINARY BIOMARKERS FOR SLE AND LUPUS NEPHRITIS

(71) Applicants: PAUL C. BOUTROS, TORONTO (CA); UNIVERSITY HEALTH NETWORK, TORONTO (CA)

(72) Inventors: JOAN E. WITHER, TORONTO (CA); PAUL R. FORTIN, TORONTO (CA); HEATHER N. REICH, TORONTO (CA); JAMES W SCHOLEY, TORONTO (CA); CAROLINA M. LANDOLT-MARTICORENA, TORONTO (CA); CARMEN AVILA-CASADO, TORONTO (CA); PAUL C. BOUTROS, TORONTO (CA)

(21) Appl. No.: 15/517,846

(22) PCT Filed: Oct. 7, 2015
(86) PCT No.: PCT/CA2015/051016

§ 371 Date: Apr. 7, 2017

Related U.S. Application Data
(60) Provisional application No. 62/060,921, filed on Oct. 7, 2014.

(51) Int. Cl. G01N 33/564

(52) U.S. Cl. CPC G01N 33/564; G01N 2800/60; G01N 2800/52; G01N 2570/00; G01N 2800/104; G01N 2800/347

(57) ABSTRACT

A SLE and/or lupus nephritis (LN) biomarker panel comprising a solid support and two or more biomarker detection agents, each biomarker detection agent specific for a corresponding target biomarker selected from a group of target biomarkers listed herein is provided; as well as methods for measuring biomarker levels, predicting, prognosing, monitoring and stratifying patients with SLE including active non-LN SLE and active LN SLE.

### CROSS-REFERENCE TO RELATED APPLICATIONS

This PCT application claims the benefit of U.S. Provisional Patent Application Ser. No. 62/060,921, filed Oct. 7, 2014, which is hereby incorporated by reference in its entirety.

### FIELD

The disclosure pertains to biomarkers associated with Systemic Lupus Erythematosus (SLE), lupus nephritis (LN) and methods and products for measuring biomarker levels, and predicting, prognosing and monitoring SLE and LN.

### BACKGROUND

Lupus nephritis (LN) is a major determinant of morbidity and mortality in Systemic Lupus Erythematosus (SLE). Variability in clinical course, underlying renal injury, and response to treatment pose therapeutic challenges. Management of LN would be served by the discovery of biomarkers that accurately reflect changes in disease activity, aiding in the prompt identification of flares and evaluation of response to therapy. Renal biopsy is the most reliable way to determine the extent and nature of renal injury, with serologic changes (anti-dsDNA antibodies) or measures of renal dysfunction (proteinuria) faltering in the diagnosis of impending flares and/or assessment of therapeutic response.

Although elevated anti-dsDNA Ab levels and hypocomplementemia associate with disease activity in cross-sectional analyses, longitudinal studies indicate that these traditional biomarkers do not distinguish between active SLE patients with and without LN, and are inconsistent at predicting impending flares. Proteinuria and other measures of renal function, also falter as accurate markers of immune-mediated renal injury. In immune-mediated membranous nephritis, renal biopsies performed prior to clinical recurrence show that there has been substantial immune injury before the onset of proteinuria. Since the immune-mediated injury is similar in LN, it is likely that reliance on proteinuria as a marker of renal inflammation leads to delayed initiation of treatment, resulting in increased renal inflammation and damage prior to treatment. Conversely, it may also lead to unnecessary prolongation or premature tapering of immunosuppressive therapy due to the persistence/resolution of urinary and/or functional abnormalities that may not reflect resolution of the inciting immunologic insult. LN-associated proteinuria frequently persists for years after renal injury, normalizing in less than 50% of patients within two years. Indeed, a repeat renal biopsy is often the only way to distinguish between persistent activity (active SLE with LN) and a chronic inactive lesion (LN patient in remission).

SLE is a complex autoimmune disease affecting approximately 1 in 1,000 individuals. There is a predilection for the kidney with 50-60% of SLE patients developing LN, with the majority developing this within the first 3 years following diagnosis. LN and its treatment result in significant immediate (e.g., infection) and delayed onset (e.g., avascular necrosis and cardiovascular disease) morbidity and mortality with up to 15% of patients developing end-stage renal disease. Typically LN runs a relapsing and remitting course, with each flare of disease increasing the risk of permanent renal damage. This renal damage is thought to result from delays in initiation of treatment reflecting the inability to detect renal inflammation before damage occurs, as well as, a relative inability to determine whether treatments have been effective until significant additional damage has occurred. Therefore biomarkers are required to determine response to therapies and early flare to decrease the significant burden of LN and its treatment. Given the enormous cost of clinical trials and difficulty in showing treatment responses before 24 months following introduction of therapy, measurement of biomarkers that reflect the extent of renal inflammation could also provide an early indication of potential therapeutic effectiveness, bolstering confidence in a potential successful outcome.

### SUMMARY

An aspect includes a SLE and/or lupus nephritis (LN) biomarker panel comprising a solid support and two or more biomarker detection agents, each biomarker detection agent specific for a corresponding target biomarker selected from a group of target biomarkers as listed in FIG. 8, in FIG. 2, in FIG. 4, in FIG. 5, in Table 1, and/or in Table 2.

Another aspect includes a method of measuring a level of two or more target biomarkers in a urine sample comprising contacting a urine sample with a biomarker panel described herein and/or two or more biomarker detection agents, each biomarker detection agent specific for a corresponding target biomarker selected from a group consisting of target biomarkers listed in FIG. 8, in Table 1, in Table 2, in FIG. 2, in FIG. 4, and/or optionally including IP-10 and/or PDGF-BB, under conditions for forming a complex between the target biomarkers in the urine sample and their corresponding biomarker detection agents; quantifying the amount of complex formed for the two or more of the target biomarkers; and optionally comparing to a control.

Another aspect includes a method of diagnostic assessment comprising measuring levels using two or more target biomarkers in a urine sample, the method comprising contacting a urine sample with a biomarker panel described herein and/or two or more biomarker detection agents, each biomarker detection agent specific for a corresponding target biomarker selected from a group consisting of target biomarkers listed in FIG. 8, in FIG. 4, in Table 1, in Table 2, in FIG. 2, in FIG. 5, optionally also including IP-10 and PDGF-BB, under conditions for forming a complex between each of the target biomarkers in the urine sample and its corresponding biomarker detection agents; quantifying the amount of complex formed for two or more of the target biomarkers; and optionally comparing to a control.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the present disclosure will now be described in relation to the drawings in which:

FIG. 1: Longitudinal changes in urinary biomarkers over time. Shown are results for certain urinary biomarkers (adiponectin, PAI-1, PF4, TIMP-1) and standard measurements (rSLEDAI, proteinuria, creatinine) for 2 patients who were treatment failures at 2 years following treatment initiation (FIG. 1A) and 2 patients who achieved a complete remission (i.e., absence of urinary changes and normal creatinine) (FIG. 1B). Note the normalization of renal parameters in patients who achieve a remission at 12-15 months, whereas this is not the case for patients who ultimately failed therapy.

FIG. 2: Representative urinary biomarkers effectively discriminate between active lupus nephritis and active non-lupus nephritis patients.

Scatter plot showing the normalized concentration of selected urinary proteins in SLE patients with active LN (n=60, open circles), active non-LN (n=25 closed circles) and healthy controls (n=24, closed squares). Urinary concentrations were corrected for creatinine to normalize for osmolality. Panel A, Representative novel urinary biomarkers identified including, membrane metalloproteinase-2 (MMP-2), plasminogen activator inhibitor-1 (PAI-1) and adiponectin. Panel B, Proposed urinary biomarkers including monocyte chemoattractant protein-1 (MCP-1), TNF-related weak inducer of apoptosis (TWEAK) and neutrophil gelatinase-associated lipocalin (NGAL). For all panels each symbol represents the determination from a single individual, with the mean value for each group indicated by a horizontal line. *** q values (≦10⁻⁴), ** (≦10⁻³) and * (10⁻²) as calculated from linear modeling summarized in FIG. 3.

FIG. 3: Urinary and not serum adiponectin is specifically associated with active LN.

Panel A: Scatter plot showing the normalized concentration of urine adiponectin in SLE patients with active renal disease (n=20, open circles) and inactive renal disease (e.g., LN patients in remission) with a prior history of biopsy-proven nephritis (n=25 closed circles). Active renal disease was defined as having 2 or more components of the renal SLEDAI-2K defined as proteinuria>0.5 gm/d, hematuria>5 RBC/hpf, pyuria>5 WBC/hpf or heme granular or RBC casts. Inactive renal disease was defined as the absence of any one of these urinary abnormalities at least 6 months prior and at the time of recruitment. Panel B: Linear correlation of plasma and urinary concentrations of adiponectin in SLE patients (n=13) with active LN.

FIG. 4: A select number of urinary proteins identify patients with proliferative renal lesions.

Scatter plot showing the normalized concentration of selected urinary proteins in SLE patients with active LN (n=60, closed circles), active non-LN (n=25, open circles), proliferative lesions (open triangles), non-profiliterative/fibrotic lesions (closed triangles) and healthy controls (n=24, closed squares). Ten urine analytes significantly correlate with activity score on renal biopsy: adiponectin, PAI-1, sgp130, IL-16, HGF, vWF, TIMP-1, Eotaxin, IP-10 and PDGF.BB. Of these 5 were noted to discriminate between proliferative and other renal lesions on renal biopsy. Analytes shown: IP-10 (units), vWF (units), Adiponectin (units), IL-16 (units) and PAI-1 (units), albumin (units). For all panels each symbol represents the determination from a single individual, with the mean value for each group indicated by a horizontal line. Significance levels were determined by Mann-Whitney non-parametric testing. Only the differences between proliferative and non-proliferative/fibrotic lesions for each analyte are shown. Statistically significant p values shown *** <0.0001, ** 0.0001, * 0.001.

FIG. 5. Results for urinary biomarkers in a discovery cohort. Scatter plot showing the normalized concentration of selected urinary proteins in SLE patients with active LN (n=60, open circles), active non-LN (n=25 closed circles) and healthy controls (n=24, closed squares). Urinary concentrations were corrected for creatinine to normalize for osmolality. Panel A, Representative novel urinary biomarkers identified including, tissue inhibitor of metalloproteinase-1 (TIMP-1), plasminogen activator inhibitor-1 (PAI-1) and adiponectin. Panel B, Previously proposed urinary biomarkers including monocyte chemoattractant protein-1 (MCP-1), TNF-related weak inducer of apoptosis (TWEAK) and neutrophil gelatinase-associated lipocalin (NGAL).

FIG. 6. Results from a validation study (Example 4). Scatter plot showing the normalized concentration of selected urinary proteins in SLE patients from the validation cohort with active LN (n=33, open circles), active non-LN (n=17 closed circles) and healthy controls (n=24, closed squares). Urinary concentrations were corrected for creatinine to normalize for osmolality. Results are shown for tissue inhibitor of metalloproteinase-1 (TIMP-1), plasminogen activator inhibitor-1 (PAI-1) and adiponectin.

FIG. 7. Results showing normalization of urinary analytes in LN patients in remission. Scatter plot showing the normalized concentration of selected urinary proteins in SLE patients. Results for 30 LN patients in remission (closed squares) are shown as compared to pooled results from the discovery and validation cohorts for patients with active LN (n=93, open circles) and active non-LN (n=42). Urinary concentrations were corrected for creatinine to normalize for osmolality. Results are shown for tissue inhibitor of metalloproteinase-1 (TIMP-1), plasminogen activator inhibitor-1 (PAI-1) and adiponectin.

FIG. 8. A distinct protein cluster discriminates between active SLE patients with and without lupus nephritis.

Linear modeling was used to examine differences in protein expression between disease states (column 1, all active SLE (n=85, with and without LN) vs healthy controls) and between disease types (column 2, active LN vs active non-LN). The bracket on the left indicates the 37 urinary analytes that are preferentially increased (except for X6CKine, which is decreased) in active LN versus active non-LN SLE patients. The 9 urinary analytes not encompassed by the bracket (CCL20.MIP.3A, CXCL6.GCP.2, CXCL11.I.TAC, CCL14a.HCC.1, CCL19.MIP.3B, sTNFRI, TIMP.1, IFN-gamma, and Beta-2-microglobulin) are significantly different (increase or decrease) in active SLE patients vs healthy controls. The colour and size of the circle indicate the directionally (increase or decrease (decreased amounts are starred)) and the magnitude (fold change) for each analyte. The colour density of the bar indicates the q value (p value corrected for multiple testing). Note that the circle shown for MMP-2 should be two-fold rather than eight-fold.

### DETAILED DESCRIPTION OF THE DISCLOSURE

In any definition of a biomarker found herein, the use of periods to separate terms within the biomarker's name may also be known in the art by the terms separated by hyphens or parentheses, e.g., “IL.16” is herein synonymous with “IL-16” (Interleukin 16). Also, for biomarkers known by more than one name in the art, these synonyms may be set off either by periods or parentheses, e.g., “CCL20.MIP.3A” is synonymous with “CCL20 (MIP.3A)”, where CCL20 (Chemokine C—C motif ligand 20) and MIP.3A (Macrophage inflammatory protein 3A) both refer to the same protein.

The term “IL-1R1” as used herein means “interleukin-1 receptor type 1”.

The term “MMP” as used herein means “matrix metalloproteinase”.

The term “PAI-1” as used herein means “plasminogen activator inhibitor-1”.

The term “vWF” as used herein means “von Willebrand factor”.

The term “MPO” as used herein means myeloperoxidase.

The term “PF4” as used herein means “platelet factor 4” which is synonymous with “CXCL4” (chemokine C—X—C motif ligand 4).

The term “SVAM-1” or “sVCAM-1” as used herein means “soluble vascular (cell) adhesion molecule 1”.

The term “GRO” as used herein means “growth related oncogene”.

The term “MCP” as used herein means “monocyte chemotactic protein”.

The term “sFas” as used herein means “soluble Fas”.

The term “SCF” as used herein means “Skp, Cullin, F-box containing complex”.

The term “HCF” as used herein means “host cell factor”.

The term “BCA-1” as used herein means “B-cell-attracting chemokine 1”.

The term “CXCL” as used herein means “chemokine C—X—C motif ligand”.

The term “KIM-1” as used herein means “kidney injury molecule-1”.

The term “TARO” as used herein means “thymus and activation related chemokine”.

The term “TWEAK” as used herein means “TNF-related weak inducer of apoptosis”.

The term “PDGF-BB” as used herein means “platelet-derived growth factor BB”.

The term “HGF” as used herein means “hepatocyte growth factor”.

The term “sgp30” or “sgp130” as used herein means “soluble glycoprotein 130”.

The term “TIMP-1” or “TIMP.1” as used herein means TIMP metallopeptidase inhibitor 1. The term “TIMP-2” or “TIMP.2” as used herein means TIMP metallopeptidase inhibitor 2.

The term “GM.CSF” as used herein means “granulocyte macrophage colony-stimulating factor”.

The term “VEGF” as used herein means “vascular endothelial growth factor”.

The term “NGAL” as used herein means “neutrophil gelatinase-associated lipocalin”.

The term “sTNFRI” as used herein means “soluble tumor necrosis factor receptor I”.

The term “active LN” as used herein means patients with active SLE who have active lupus nephritis.

The term “active non-LN” as used herein means patients with active SLE who do not have lupus nephritis or a history of lupus nephritis.

The term “LN patients in remission” as used herein means a subject with a history of LN (e.g biopsy proven LN) and without active disease, for example having a serum creatinine within 10% of age-related upper limit of normal and urine protein: creatinine<25 mmol/umol.

The term “SLEDAI-2K” as used herein means the validated and published SLE disease activity-2000 index.¹⁰

The term “SLE biomarker” as used herein means a biomarker selected from CCL20.MIP.3A, CXCL6.GCP.2, CXCL11.I.TAC, CCL14a.HCC.1, CCL19.MIP.3B, sTNFRI, TIMP.1, IFN-gamma, and Beta-2-microglobulin. It was found that of the SLE biomarkers, CCL20.MIP.3A, CXCL6.GCP.2, CCL14a.HCC.1, sTNFRI and IFN-gamma were decreased in subjects with SLE and the remaining SLE biomarkers were increased relative to healthy controls.

The term “SLE biomarker” or “SLE biomarker set” as used herein means one or more biomarkers selected from CCL20.MIP.3A, CXCL6.GCP.2, CXCL11.I.TAC, CCL14a.HCC.1, CCL19.MIP.3B, sTNFRI, TIMP.1, IFNgamma, and Beta-2-microglobulin.

The term “LN biomarker” as used herein means a biomarker selected from CXCL6.GCP.2, CXCL11.I.TAC, sTNFRI, TIMP.1, IFNgamma, CXCL7.NAP.2, Adiponectin, PAI.1, sVCAM.1, TWEAK, sgp130, sIL.1RI, KIM.1, Albumin, Clusterin, CystatinC, Eotaxin.2, BCA.1, IL.16, TARC, X6CKine, SCF, HGF, SAP, PF4.CXCL4, vWF, Myeloperoxidase, sFas, Perforin, MMP.2, MMP.7, MMP.9, TIMP.2, Eotaxin, GM.CSF, GRO, MCP.3, IL.15, IL.6, IL.8, MCP.1, VEGF, IP-10, CCL14a.HCC.1, NGAL, and PDGF-BB.

The term “LN biomarker set” as used herein means one or more biomarkers selected from CXCL6.GCP.2, CXCL11.I.TAC, sTNFRI, TIMP.1, IFNgamma, CXCL7.NAP.2, Adiponectin, PAI.1, sVCAM.1, TWEAK, sgp130, sIL.1RI, KIM.1, Albumin, Clusterin, CystatinC, Eotaxin.2, BCA.1, IL.16, TARC, X6CKine, SCF, HGF, SAP, PF4.CXCL4, vWF, Myeloperoxidase, sFas, Perforin, MMP.2, MMP.7, MMP.9, TIMP.2, Eotaxin, GM.CSF, GRO, MCP.3, IL.15, IL.6, IL.8, MCP.1, VEGF, IP-10, CCL14a.HCC.1, NGAL, and PDGF-BB, with the proviso that the selection cannot be solely albumin, adiponectin, or NGAL.

The term “reduced biomarker set” as used herein means one or more biomarkers selected from adiponectin, PAI-1, vWF, TIMP-1, IL-15, PF4, sVCAM-1, and NGAL, with the proviso that the selection cannot be solely albumin, adiponectin, or NGAL.

The term “screening biomarker set” as used herein means one or more biomarkers selected from adiponectin, PAI-1, vWF, and NGAL, with the proviso that the selection cannot be solely adiponectin, or NGAL.

The term “one or more” as used herein means 1, 2, 3 etc upto the number of elements of the set, Table or Figure. For example one or more biomarkers of the “screening biomarker set” includes any one (with the proviso that the selection cannot be solely albumin, adiponectin, or NGAL), any two, any three or all 4 of adiponectin, PAI-1, vWF, and NGAL.

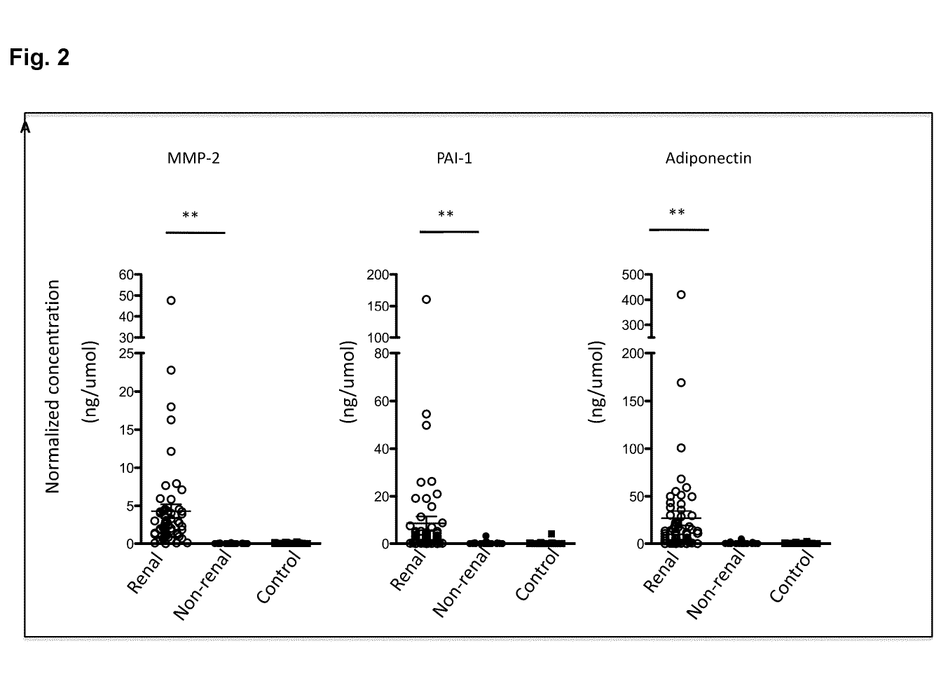

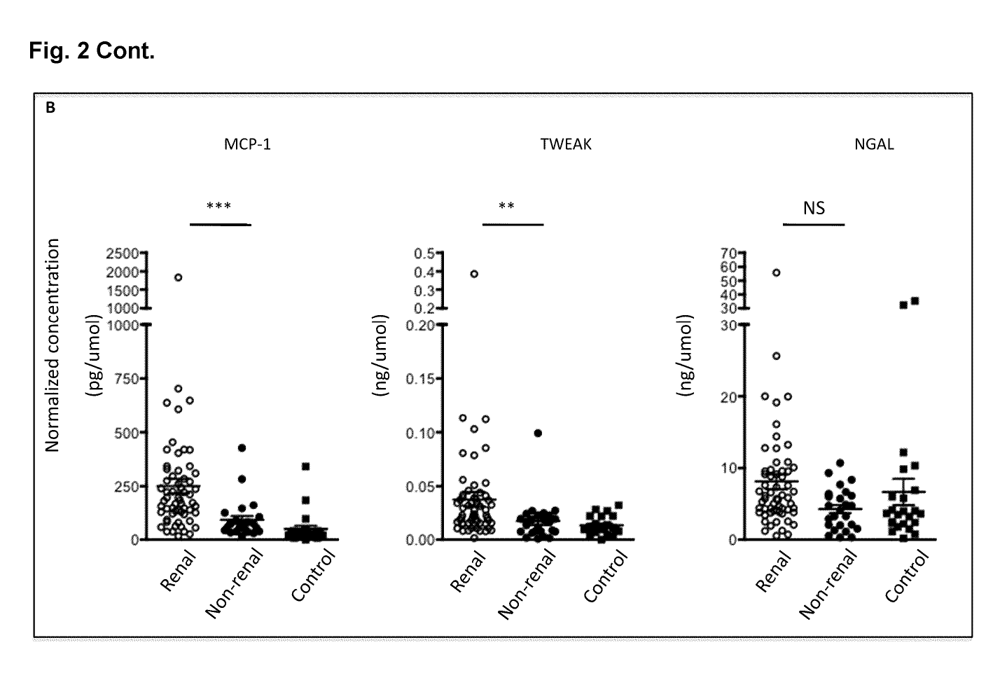

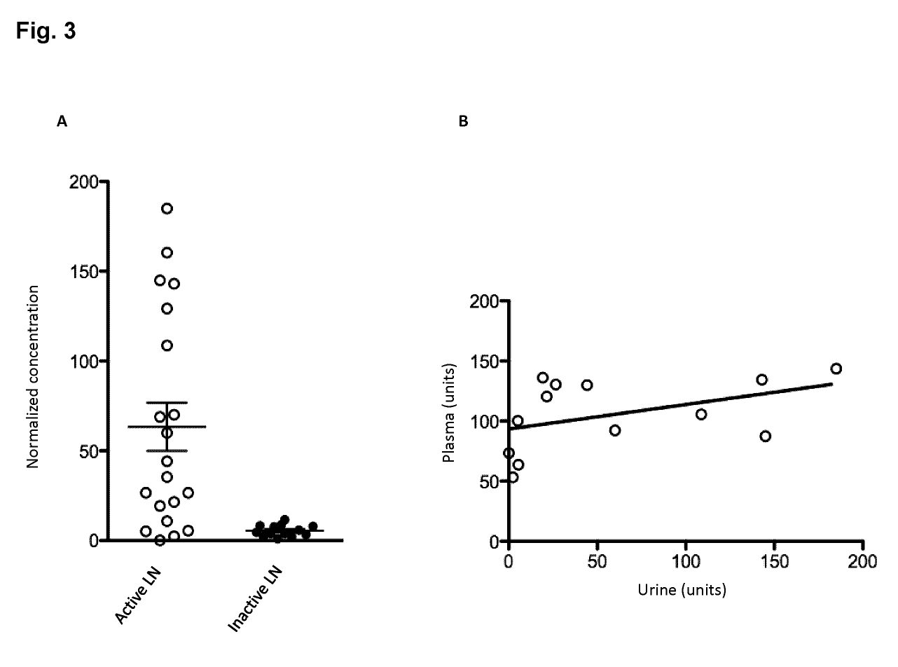

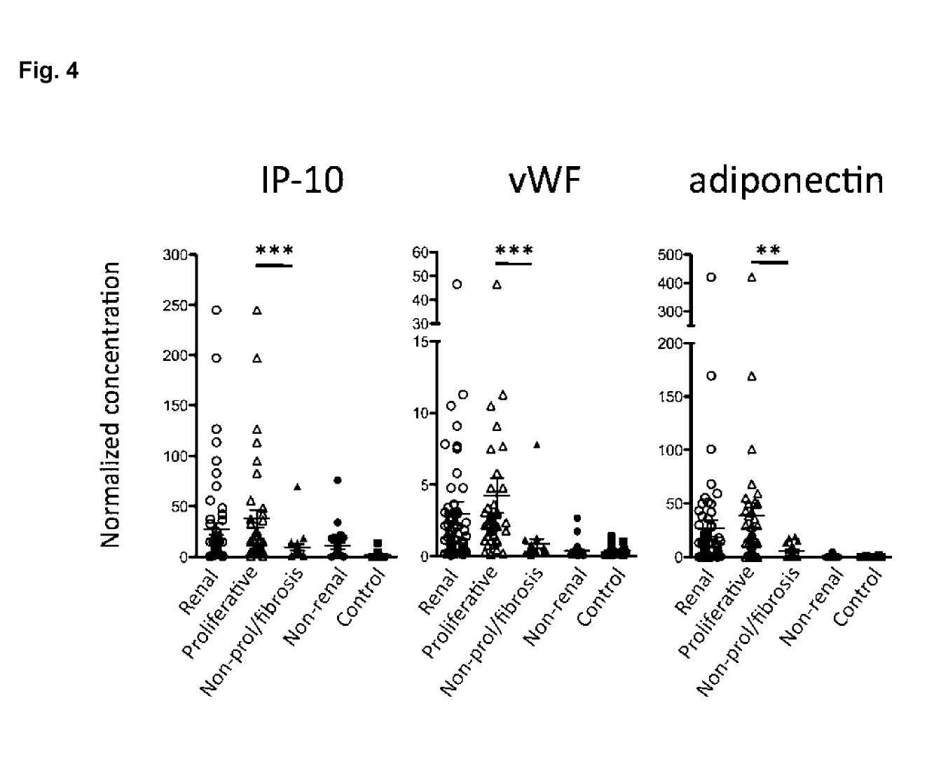

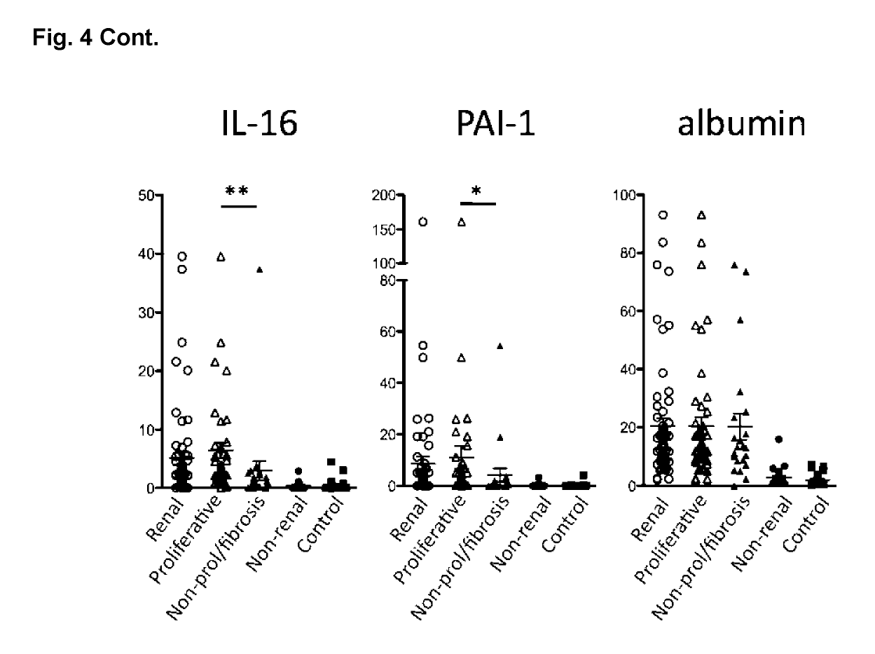

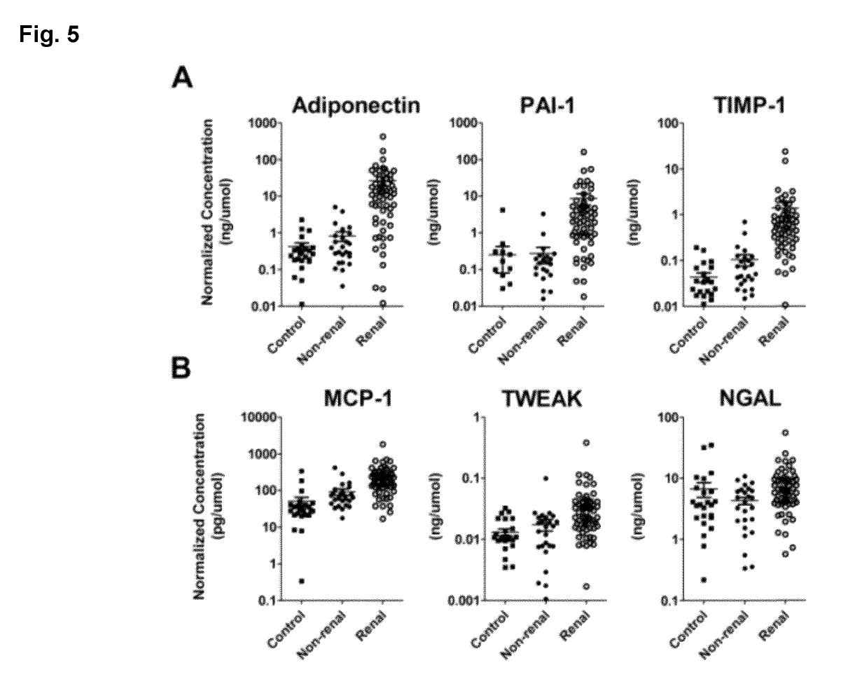

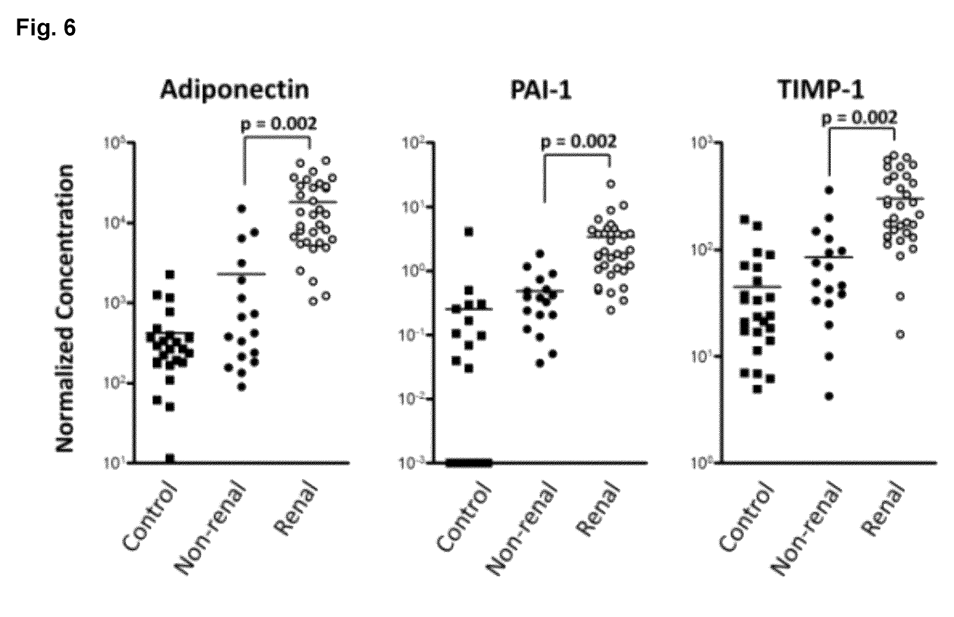

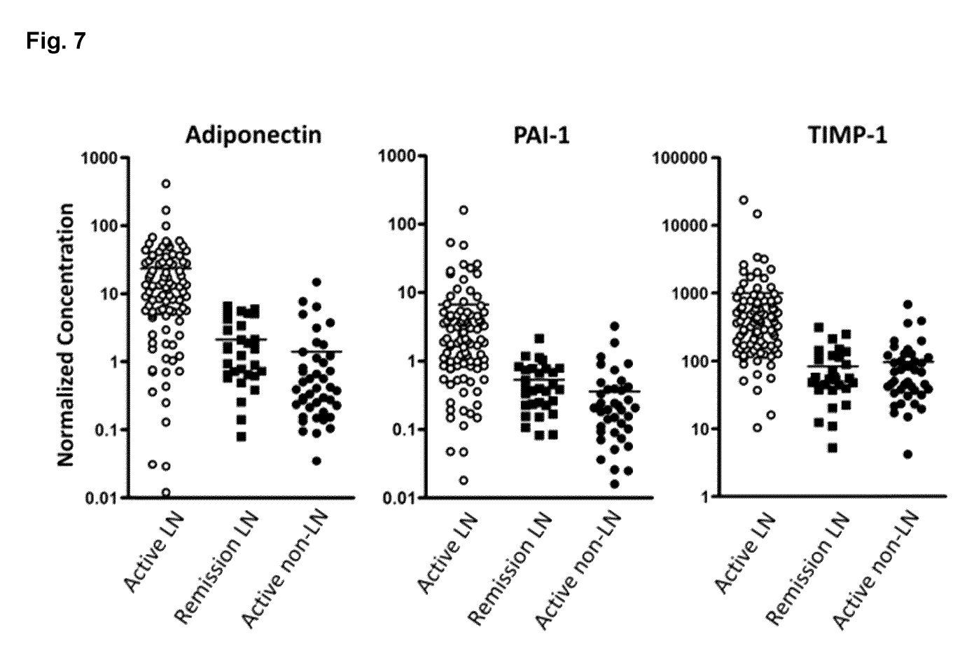

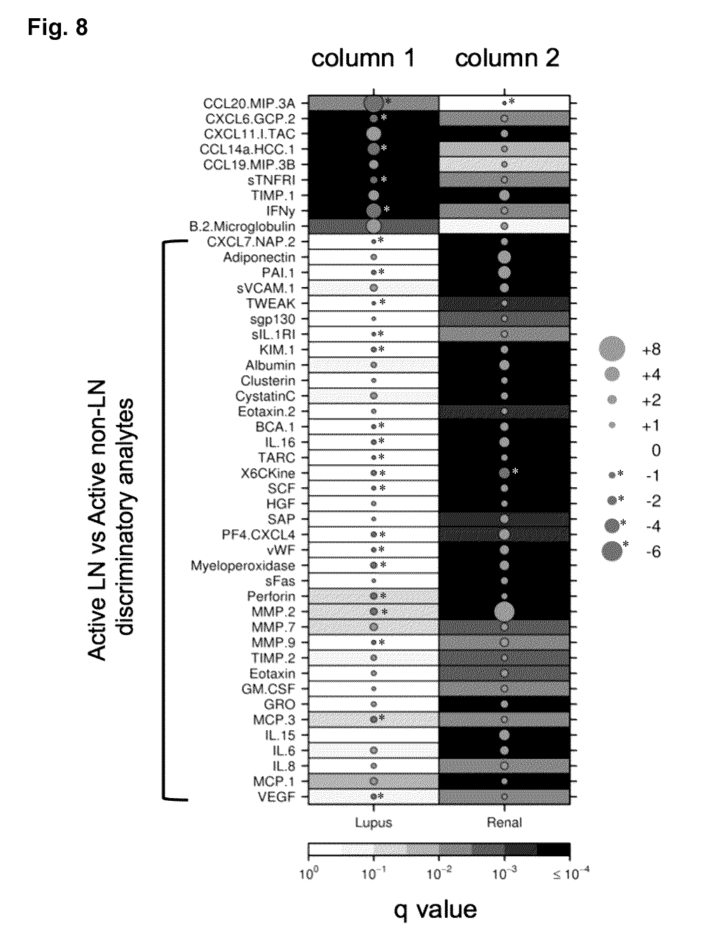

The term “control” as used herein is sample obtained from a subject without a condition being assessed. The control can also be a reference value. The reference value is determined for each biomarker by reference to a preselected value such as an average level or median level exhibited in a clinical population that does not exhibit the condition or disease to be detected. In methods for monitoring disease, the reference level can be a prior level of the subject. The reference value for a biomarker can for example be determined by reference to the corresponding control values depicted in FIGS. 2, 4, 5, 6, and/or 7 and/or determined by similar methods as described herein in other control populations.

The term “subject control” as used herein refers to an earlier sample or base line level. For example, in methods for assessing if a subject is responding to a therapy, it the subject's disease is progressing or ameliorating, the sample is compared to the subject's previous test result or sample. For biomarkers whose level increases with for example active proliferative disease, a decrease in the subject sample compared to the subject control indicates that the subject is responding to treatment (if receiving treatment) or that the active proliferative disease is resolving.

An aspect includes a SLE and/or lupus nephritis (LN) biomarker panel comprising a solid support and two or more biomarker detection agents, each biomarker detection agent specific for a corresponding target biomarker selected from a group of target biomarkers as given in a foregoing defined set, and/or in FIG. 8, FIG. 2 herein, Table 1 herein, Table 2 herein, FIG. 4 herein, and/or FIG. 5 herein. The two or more biomarker detection agents can for example be from the same set or from different sets.

In an embodiment, the biomarker panel comprises or consists of 2, 3, 4, 5, 6, 7, 8, 9, 10, 11, 12, 13,14, 15, 16, 17, 18, 19, 20, 21, 22, 23, 24, 25, 26, 27, 28, 29, 30, 31, 32, 33, 34, 35, 36, 37, 38, 49, 40, 41, 42, 43, 44, or 45 biomarker detection agents, each specific for a corresponding target biomarker. In an embodiment, the biomarker panel comprises multiple biomarker detection agents for each specific biomarker. For example, a biomarker panel may comprise 20 biomarker detection agents with two biomarker reagents specific for each of 10 different biomarkers, for example where the biomarker reagents are located on different areas of a panel plate to reduce positional discrepancies. All combinations of biomarker detection agents (e.g. different combinations from the same Tables and/or Figures as well as different combinations from different Tables and figures described herein) are contemplated.

In an embodiment, each biomarker detection agent is an antibody or binding fragment thereof.

The term “antibody” as used herein is intended to include monoclonal antibodies, polyclonal antibodies, chimeric antibodies, humanized antibodies as well as human antibodies, identified for example using phage display, and antibody binding fragments thereof. The antibody may be from recombinant sources and/or produced in transgenic animals. The term “antibody binding fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

In an embodiment, the detection agent is a soluble receptor or an aptamer specific for a biomarker described herein.

In an embodiment, the antibody is a monoclonal antibody.

In an embodiment, the antibody is labelled with a detectable label. The detectable label can be a directly detectable label or an indirectly detectable label. For example, the antibody can be labelled with a fluorescent label, biotin, comprise a radioactive moiety.

In an embodiment, the target biomarker is selected from the group consisting of CCL20.MIP.3A (e.g. CCL20 and MIP.3A are different names used to refer to the same protein), CXCL6.GCP.2, CXCL11.I.TAC, CCL14a.HCC.1, CCL19.MIP.3B, sTNFRI, TIMP.1, IFNy, Beta-2-microglobulin. As described in Example 1, these are the 9 biomarkers that distinguished SLE and healthy controls. These biomarkers were significantly increased or decreased between at least 2 to upto 8 fold in subjects with SLE versus healthy controls as shown in FIG. 8.

In an embodiment, the target biomarker is selected from the group consisting of CXCL6.GCP.2, CXCL11.I.TAC, sTNFRI, TIMP.1, IFNgamma, CXCL7.NAP.2, Adiponectin, PAI.1, sVCAM.1, TWEAK, sgp130, sIL.1RI, KIM.1, Albumin, Clusterin, CystatinC, Eotaxin.2, BCA.1, IL.16, TARC, X6CKine, SCF, HGF, SAP, PF4.CXCL4, vWF, Myeloperoxidase, sFas, Perforin, MMP.2, MMP.7, MMP.9, TIMP.2, Eotaxin, GM.CSF, GRO, MCP.3, IL.15, IL.6, IL.8, MCP.1, VEGF, IP-10, CCL14a.HCC.1, NGAL, and PDGF-BB.

In an embodiment, the solid support is a bead, a well plate, or chip.

In an embodiment, the bead comprises a unique code optionally a colour code and each unique code is associated with each biomarker detection agent.

In an embodiment, the biomarker panel includes two or more biomarker detection agents each that specifically bind a SLE and/or LN biomarker.

A further aspect includes a kit comprising the biomarker panel described and/or two or more biomarker detection agents, each biomarker detection agent specific for a corresponding target biomarker selected from a set described herein; and

one or more of:

sample dilution buffer;

wash buffer;

filter;

positive control; and/or

instructions for performing a method described herein.

In an embodiment, the biomarker detection agents are selected from detection agents that correspond to a biomarker as given in a foregoing defined set, and/or in FIG. 8, FIG. 2 herein, Table 1 herein, Table 2 herein, FIG. 4 herein, and/or FIG. 5 herein.

The kit can comprise for example 2, 3, 4, 5, 6, 7, 8, 9, 10, 11, 12, 13,14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 biomarker detection agents.

A further aspect is a method of measuring level two or more target biomarkers in a urine sample, the method comprising contacting a urine sample with a biomarker panel described herein and/or two or more biomarker detection agents, each biomarker detection agent specific for a corresponding target biomarker selected from a group consisting of target biomarkers listed in FIG. 8, FIG. 2 herein, Table 1 herein, Table 2 herein, FIG. 4 herein, FIG. 5 herein, IP-10 and/or PDGF-BB, under conditions for forming a complex between each of the target biomarkers in the urine sample and each of their corresponding biomarker detection agents; quantifying the amount of complex formed for two or more of the target biomarkers, thereby measuring the levels of the two or more biomarkers; and optionally comparing to a control.

Another aspect includes a method of detecting a level of two or more target biomarkers in a urine sample comprising contacting a urine sample with a biomarker panel described herein and/or two or more biomarker detection agents, each biomarker detection agent specific for a corresponding target biomarker selected from a group consisting of target biomarkers listed in FIG. 8, in Table 1, in Table 2, in FIG. 2, in FIG. 4, and/or optionally including IP-10 and/or PDGF-BB, under conditions for forming a complex between the target biomarkers in the urine sample and their corresponding biomarker detection agents; quantifying the amount of complex formed for the two or more of the target biomarkers; and optionally comparing to a control.

In an embodiment, the urine sample is obtained at the time of diagnostic renal biopsy. In other embodiments, the urine sample is obtained at multiple intervals.

As above, the two or biomarker detection agents can for example be from the same set or from different sets (e.g. from the same Table and/or Figure or different Tables and/or Figures described herein). In an embodiment, the method comprises or consists of measuring the levels of 2, 3, 4, 5, 6, 7, 8, 9, 10, 11, 12, 13,14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 biomarker detection agents, each specific for a corresponding target biomarker. In an embodiment, the biomarker panel comprises multiple biomarker detection agents for a specific biomarker. For example, a biomarker panel may comprise 20 biomarker detection agents with two biomarker reagents specific for each of 10 different biomarkers, for example where the biomarker reagents are located on different areas of a panel plate to reduce positional discrepancies. All combinations of biomarker detection agents (e.g. different combinations from the same Tables and/or Figures as well as different combinations from different Tables and figures described herein) are contemplated.

In an embodiment, the urine sample is obtained from a subject with Systemic Lupus Erythematosus (SLE) or suspected of having SLE.

In an embodiment, the SLE is childhood-onset SLE (cSLE). In an embodiment the SLE is adult SLE.

In an embodiment, the method is for diagnosing SLE and a fold increase observed in the level of one or more target biomarkers listed in the SLE biomarker set (as given, e.g., in FIG. 8 and/or Table 1) to be increased compared to a control and/or a fold decrease observed in the level of one or more target biomarkers listed in the SLE biomarker set to be decreased compared to a control indicates the subject has SLE.

In an embodiment, method is for monitoring disease activity, optionally for identifying exacerbation of LN or onset of new LN, and/or stratifying patient with regards to extent of renal injury, and optionally wherein the one or more target biomarkers are selected from the LN biomarker set or the reduced biomarker set or the screening biomarker set, optionally wherein the method further comprises treating the subject and/or intensifying treatment according to the disease activity.

In an embodiment, the method is for distinguishing SLE with active LN from SLE active without LN (e.g. active non-LN), and optionally the one or more target biomarkers are selected from the LN biomarker set or the reduced biomarker set or the screening biomarker set.

In an embodiment, the urine sample is obtained from a subject that has received or is receiving treatment for LN and a decrease in one or more target biomarker levels shown to be increased in Table 1 or Table 2 compared to a control indicates the subject has responded or is responding to the treatment.

For example the panel, detection agents and methods described herein can be used for disease and treatment monitoring, prognosing disease outcome (i.e. in the absence or presence of treatment). For example detecting an increase in one or more SLE biomarkers listed in the SLE biomarker set as elevated compared to a control indicates the subject has or has an increased likelihood to develop SLE and a lack of increase indicates the subject does not have and/or is unlikely to develop SLE. Also in a further example, detecting an increase in one or more LN biomarkers listed in Table 1 or Table 2 as elevated compared to a control indicates the subject has or has an increased likelihood to develop LN. Appropriate treatment can be initiated. The methods described herein can also be used to monitor a LN flare and/or monitor response to a treatment. If a treatment is not working it can be modified, stopped and/or an alternative intervention can be taken.

In embodiments monitoring treatment response and/or disease progression, the method can be repeated, for example after about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months after a flare or the initiation of treatment. In embodiments for monitoring treatment, or monitoring progression or regression, the subject value is compared to for example either a control such as a population reference value or an earlier sample or reference level of the subject (i.e. a subject control).

In an embodiment, the treatment is part of the standard of care when a patient has been diagnosed with active lupus nephritis. For example, the method can be used to identify patients with active lupus nephritis. In an embodiment, the method further includes prescribing prednisone and an immunosuppressant. In an embodiment, the treatment is part of the standard of care for continued management of active lupus nephritis. For example, the method can be used to determine whether tapering of prednisone has resulted in increased nephritis as compared to the previous measurement of biomarker levels. In an embodiment, the method further includes changing the dose of prednisone or prescribing a different immunosuppressant. Immunosuppressant drugs used for the treatment and/or management of LN include plaquenil, imuran, mycofenolate, and cyclophosphamide.

In an embodiment, the treatment is a test treatment. For example, the subject can be in a clinical trial and the method is used to identify early responders to the study treatment. In an embodiment, the test treatment is selected from tacrolimus, cyclosporine A, abatacept, fludaribine, deoxyspergulin, rituximab, and therapeutics working by the same or similar mechanisms of action.

In an embodiment, the biomarker level is used to identify a clinical response to a treatment. For example, clinical response can be renal response; remission defined as a serum creatinine within 10% of age-related upper limit of normal and urine protein: creatinine<25 mmol/umol; partial response defined as a return of eGFR to within 50% of pre-flare level and a >50% reduction in proteinuria to under 1 g/day (urine protein:creatinine<75 mmol/umol).

In an embodiment, one or more target biomarkers are used for assessing whether the subject is in remission. For example, the one or more biomarkers can be selected from PAI.1, IL.15, PF4.CXCL4, TIMP.1, vWF, sVCAM.1, IL.6, KIM-1, GRO, Cystatin C, CXCL6.GCP.2, Clusterin, MMP.7, MCP.1, and GM.CSF. It is demonstrated for example that the aforementioned target biomarkers are also significantly lower in LN patients that were in remission as compared to patients with active LN.

In an embodiment, the biomarker panel is used for predicting long-term response to therapy.

In an embodiment, the predictive composite panel (e.g. LN-treatment response) is used alone or in combination with clinical parameters such as creatinine and proteinuria.

In an embodiment, the one or more target biomarkers is/are selected from MMP-2, plasminogen activatory inhibitor-1 (PAI-1) and/or adiponectin.

In an embodiment, the one or more target biomarkers is/are selected from MCP-1, NGAL, and/or TWEAK.

In an embodiment, one or more target biomarkers is/are adiponectin.

It is demonstrated herein that one or more biomarkers correlate with activity score on renal biopsy and can be used for example to stratify the subjects according to severity of flare.

In an embodiment, the one or more target biomarkers is/are selected from Adiponectin, PAI.1, IL.15, PF4.CXCL4, TIMP.1, albumin, vWF, sVCAM.1, IL.6, KIM-1, GRO, Cystatin C, CXCL6.GCP.2, IL.8, Clusterin, MMP.7, MCP.1, and GM.CSF.

In an embodiment, the one or more target biomarkers is/are selected from Adiponectin, PAI.1, IL.15, PF4.CXCL4, TIMP.1, vWF, sVCAM.1, IL.6, KIM-1, GRO, Cystatin C, CXCL6.GCP.2, Clusterin, MMP.7, MCP.1, and GM.CSF.

In an embodiment, the one or more target biomarkers is/are selected from adiponectin, PAI-1, sgp130, IL-16, HGF, vWF, TIMP-1, Eotaxin, IP-10 and PDGF.BB.

In another embodiment, the one or more target biomarkers is/are selected from adiponectin, PAI-1, IL-16, vWF, Eotaxin, IP-10 and PDGF.BB. In another embodiment, the one or more target biomarkers is/are selected from IP-10, vWF, adiponectin, IL-16 and PAI-1. In yet another embodiment, the one or more target biomarkers is/are selected from vWF, PAI-1, and adiponectin.

In an embodiment, the one or more target biomarkers is/are selected from adiponectin, PAI-1, vWF, TIMP-1, IL-15, PF4, sVCAM-1, and NGAL.

In an embodiment, the one or more target biomarkers is/are selected from adiponectin, PAI-1, vWF, and NGAL.

In an embodiment, the one or more of these markers for example selected from adiponectin, PAI-1, IL-16, vWF, Eotaxin, IP-10 and PDGF.BB or selected from IP-10, vWF, adiponectin, IL-16 and PAI-1 or selected from vWF, PAI-1, and adiponectin are used for discriminating active proliferative and non-proliferative chronic renal lesions and/or for identifying subjects with active proliferative renal lesions.

As mentioned, it is also demonstrated herein that one or more biomarkers can discriminate between proliferative and other renal lesions on renal biopsy. As different treatments are available, determining the levels of these biomarkers can be used to select a treatment.

In an embodiment, an increased level compared to a control in one or more of the target biomarkers is indicative of active proliferative renal lesions.

In an embodiment, the method further comprises administering an active proliferative lesion suitable treatment if an increased level of one or more of the biomarkers is detected and a non-proliferative/chronic lesion suitable treatment if a lack of increased level of one or more of the biomarkers is detected. In an embodiment, 2, 3, 4, 5, 6, 7, 8, 9, 10, 11, 12, 13,14, 15, 16, 17, 18, 19, 20, 21, 22, 23, 24,25, 26,27, 28, 29, 30, 31, 32, 33, 34, 35, 36, 37, 38, 39, 40, 41, 42, 43, 44, and/or 45 target biomarker levels are measured.

In an embodiment, one or more of the biomarkers is increased or decreased by at least about 25%, about 50%, about 75%, about 100%, or at least about 2, about 3, about 4, about 5, about 6, about 7 or about 8 fold.

In an embodiment, the biomarker level is a standardized level, optionally standardized to creatinine. Standardization to creatinine can for example include measuring the urinary creatinine level and dividing the subject biomarker level by the subject's creatinine level.

In another embodiment, the biomarker level is not standardized. For example, large differences in measurements compared to a control may not need to be standardized.

In an embodiment, one or more SLE or LN clinical markers are also assessed, optionally eGFR and/or proteinuria.

A variety of immunoassays can be used including for example Western blot, tissue immunohistochemistry, ELISA, and arrays as well as multiplex assays such as bead based multiplex assays.

In an embodiment, biomarker levels are measured for multiple biomarkers using a machine capable of multiplex detection and quantification, such as the Bio-plex 200 suspension array system (Bio-Rad Laboratories) or the Bio-plex MAGPIX multiplex reader. In an embodiment, a suspension array system has one or more lasers, high-throughput fluidics, and digital signal processing embodied in hardware and/or software. In an embodiment, a suspension array system or multiplex reader uses Luminex-type color-coded bead sets to distinguish assay readouts, e.g., as described in U.S. Pat. Nos. 5,981,180, 6,411,904, 6,449,562, 6,658,357, 7,047,138, 8,031,918, 8,296,088, 8,274,656, 8,532,351, 8,542,897, 8,798,951, 8,859,996, 8,889,347, and 9,063,088.

In an embodiment, the control is a reference value determined for each biomarker of a particular biomarker set. In an embodiment, the reference value is determined for each biomarker by reference to average levels exhibited in a clinical population that does not exhibit the condition or disease to be detected. In an embodiment, the reference value is determined for a biomarker by reference to the corresponding control values depicted in FIGS. 2, 4, 5, 6, and/or 7. In an embodiment, the reference value as determined by the methods described herein can be further adjusted according to expected variability in a larger population.

In an embodiment, a disease or condition is detected when one or more biomarkers have a level that is above the corresponding reference value. In an embodiment, a disease or condition is detected when one or more biomarkers in a biomarker set have a level that is about two-fold higher than the corresponding reference value, or about three-fold higher, or about four-fold higher, or about five-fold higher, or about six-folder higher, or about seven-fold higher, or about eight-fold higher. In an embodiment, a disease or condition is detected when the levels a measured for a biomarker set and detection takes place when the following mathematical relation is true:

Σbᵢ/i≧zX

where i is the number of biomarker levels,
Σbᵢ is the sum over each biomarker level fold increase bᵢ,
z is optionally one, and
X is the arithmetic mean of the expected biomarker level fold increases as indicated, for example, in Table 2 for each biomarker level.

In an embodiment, diagnosis of proliferative LN (e.g. LN with active proliferative renal lesions) is made according to the aforementioned mathematical relation compared to a value determined from urine biomarker levels. For example, when the screening biomarker set is measured (e.g., where b₁₋₄ are adiponectin, PAI-1, vWF, and NGAL, respectively), i is four, and X is the arithmetic mean of 4, 3.77, 2.5, and 0.95 respectively, such that if b₁₋₄ were 5, 3.5, 2, and 1.2, diagnosis would be made since 2.925≧2.805. In an embodiment, z is set to a number between about 0.6 and about 1.4 so as to tailor diagnosis to the desired false positive or negative rate. In an embodiment, i is four, and the entirety of the screening biomarker set is measured. In an embodiment, i is eight, and the entirety of the reduced biomarker set is measured.

Also provided in another aspect is a system for generating a report diagnosing an individual, identifying for an individual with exacerbation of LN or onset of new LN, and/or stratifying patient with regards to extent of renal injury, comprising: a. a machine capable of multiplex detection and quantification such as a clinical flow cytometry multiplex device (e.g Luminex) configured to assay two or more biomarker targets in a urine sample from the individual, optionally using a biomarker panel described herein, to determine biomarker profile test values for the two or more biomarker targets, wherein the two or more biomarker targets are selected from biomarkers described herein; b. at least one computer database comprising: i. a reference value for each of the two or more biomarker targets, optionally the reference value in FIGS. 2, 4, 5, 6, and/or 7; c. a computer-readable program code comprising instructions to input the biomarker profile test values to compare each of the biomarker profile test values with a corresponding reference value from the at least one computer database in (b)(i); and d. a computer-readable program code comprising instructions to generate a report that comprises a listing of the biomarker targets for which the comparison to the reference value indicated an exacerbation of LN or onset of new LN, and/or that stratifies the individual with regards to extent of renal injury. In an embodiment, the clinical flow cytometry multiplex device is a hand held device. In an embodiment, the database (b) comprises ii. an expected biomarker fold increase for each of the two or more biomarker targets, optionally as recorded in Table 2; and the code (c) comprises instructions to input the biomarker profile test values to compare each of the biomarker profile test values with the expected biomarker fold increases from the at least one computer database in (b)(ii), optionally according to the mathematical relation given above.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5%, optionally ±10%, and/or up to ±25% of the modified term if this deviation would not negate the meaning of the word it modifies.

As used in this specification and the appended claims, the singular forms “a”, “an” and “the” include plural references unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

Further, the definitions and embodiments described are intended to be applicable to other embodiments herein described for which they are suitable as would be understood by a person skilled in the art. For example, in the above passages, different aspects of the invention are defined in more detail. Each aspect so defined can be combined with any other aspect or aspects unless clearly indicated to the contrary. In particular, any feature indicated as being preferred or advantageous can be combined with any other feature or features indicated as being preferred or advantageous.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

Methods:

Urine was obtained from 60 LN patients within 2 weeks of biopsy, 25 active non-LN SLE patients, and 24 controls. The mean age and proportion of females (83-88%) was similar in the 3 groups. 128 distinct analytes were quantified by Luminex and normalized by scaling to urinary creatinine levels. Data was analyzed by hierarchical clustering using divisive analysis (DIANA), linear modeling, and non-parametric statistics, with appropriate corrections for multiple comparisons.

Results:

LN and non-LN SLE patients had comparable SLEDAI-2K scores (13.8±7.6 and 11.1±3.9, respectively), with the majority of the SLEDAI-2K in LN patients arising from the renal indices (renal SLEDAI=8±4.3). The distribution of the renal biopsy classes (ISN-RPS) for the LN patients was: I-1; II-3, III or III/V-12; IV or IV/V-32; V-9; VI-2, TIN-1. The mean biopsy activity and chronicity scores biopsy were 6.68 (range 0-19) and 3.13 (range 0-10), respectively. Following hierarchical clustering, significant clustering was seen for LN as compared to non-LN SLE patients and healthy. Linear modeling was used to determine the urinary proteins whose abundance differed significantly between disease states (SLE vs healthy control) and between the presence or absence of LN (active LN vs active non-LN). There were 9 analytes that differed significantly (q value<0.01) between SLE patients and controls and 42 between LN and non-LN, of which 37 differed only in LN patients as compared to active non-LN patients (ie. not between SLE patients and controls). A number of proteins, not previously proposed as urinary LN biomarkers, and known candidate LN biomarkers (e.g., adiponectin), were identified, with several of the novel biomarkers showing an enhanced ability to discriminate between LN and non-LN patients over potential biomarkers reported in the literature. Ten proteins were found to significantly correlate with the activity score on renal biopsy, 7 of which (Eotaxin, PDGF-BB, IP-10, vWF, adiponectin, IL-16 and PAI-1) strongly discriminated between active proliferative and non-proliferative/chronic renal lesions, even outperforming proteinuria (albumin/creatinine ratio) in the identification of patients with active proliferative renal lesions; see FIG. 4.

Using a proteomics approach promising urinary biomarkers that correlated with the presence of active renal disease and/or renal biopsy changes were identified.

### Example 2

Given the relapsing and remitting nature of lupus nephritis (LN) and its heterogeneity with regards to the nature of renal involvement, extent of damage and response to treatment, there is enormous interest in the development of biomarkers that accurately predict these variations. To date, no suitable markers have been identified for the diagnosis, monitoring or prognosis of LN.

Utilizing a broad-based discovery approach a 42-analyte urine based signature that was developed that effectively discriminates between patients with active LN and active non-LN. A discrete analyte signature that correlates with active proliferative lesions on renal biopsy was also identified.

Care of LN patients aims to calibrate treatment to establish optimal control of inflammation and tissue injury whilst limiting exposure to immunosuppressive therapies and their attendant side effects. The clinical course of LN is marked by unpredictable flares and variable response to treatment and, in the absence of suitable biomarkers to herald disease onset or monitor early response to therapy, patient care is compromised. A renal biopsy is essential to confirm diagnosis and establish the ISN/RPS histopathological classification of the underlying renal lesion³. Histopathological differences have significant clinical implications since specific classes (e.g. proliferative vs non-proliferative) require different therapeutic interventions and are associated with divergent prognoses. To date no biomarker panel has been identified that can replace invasive renal biopsy.

Following a diagnostic renal biopsy, the monitoring of LN relies on serological biomarkers, including anti-dsDNA antibodies (Ab) and serum complement, and measures of renal dysfunction (e.g., proteinuria and measures of renal function)⁴. Although elevated anti-dsDNA Ab levels and hypocomplementemia were associated with disease activity in cross-sectional analyses, longitudinal studies indicated that these traditional biomarkers performed inconsistently in identifying active disease or predicting impending flares⁵. A prospective study of patients with newly diagnosed LN showed that serological abnormalities at renal relapse were less pronounced than at initial diagnosis and that LN flares could not be predicted prior to an obvious relapse⁵.

In addition, measures of renal dysfunction (such as proteinuria) are relatively insensitive to early immune-mediated renal injury, resulting in significant tissue inflammation and damage prior to clinical confirmation and treatment of a flare. The inability to forecast impending LN flares delays initiation of treatment, increasing the risk of poor clinical outcomes. Of equal concern is the unnecessary prolongation of immunosuppressive therapy due to the persistence of urinary abnormalities (e.g., proteinuria) without objective evidence of ongoing immune mediated injury. LN-associated proteinuria frequently persists for years after renal injury, with normalization of this urinary parameter occurring in less than 50% of patients within two years⁶.

It has been proposed that in immune-mediated renal diseases, including LN, the injury precedes the development of proteinuria and that the inciting immunological insult remits prior to improvement in proteinuria. Support for this concept stems from the study of immune-mediated membranous nephritis. Renal biopsies performed on patients with membranous nephritis prior to clinical recurrence showed that the immune injury was present prior to the onset of proteinuria⁷. Conversely, disappearance of the inciting antibody preceded resolution of proteinuria and predicted response to therapy⁸. It has been proposed that the persistent proteinuria in this condition is an indicator of healing of the renal damage rather than ongoing antibody-mediated injury⁹. A similar phenomenon potentially occurs in LN with immune-mediated injury preceding clinical evidence of renal injury and with resolution of the immune abnormalities occurring prior to the resolution of proteinuria following treatment. We propose that the interval period between changes in immunological activity and overt clinical transitions (changes in proteinuria) offers an opportunity for therapeutic intervention (intensify or reduce therapy) in anticipation of the clinical event.

As proteinuria is clinically utilized to guide therapeutic decisions, delayed improvement in urinary abnormalities prolongs immunosuppresion, increasing the risk of treatment-associated complications. In addition, the persistence of proteinuria may also reflect fixed renal damage rather than ongoing immune-mediated injury. Often, a repeat renal biopsy is the only way to distinguish between a persistent activity and a chronic inactive lesion. Therefore there is a clear need for the development of biomarkers that would (1) assist in monitoring disease activity and response to therapy, (2) accurately stratify patients with regards to extent and nature of renal injury and/or (3) inform long-term prognosis.

To date no suitable biomarkers that predict early response to therapy or forecast renal flares have been identified. This knowledge gap directly hampers the timely reduction or intensification of therapy in response to changes in disease activity, contributing to the morbidity and mortality arising from LN or its treatment. The need for biomarkers that anticipate response to treatment is particularly acute. Therapeutic clinical trials in LN often fail to reach their primary end-points within the proscribed time as current clinical indicators of disease (e.g., proteinuria) often persist beyond the length of the study. The current limitations with respect to monitoring treatment efficacy and response requires clinical trials with large study populations over a long period of time often making these studies prohibitively costly and not feasible in SLE.

Results.

An exploratory proteomic approach was used to analyze 128 urinary analytes in 60 active LN patients, 25 active non LN patients and 24 healthy controls utilizing an addressable bead system (Luminex) and results were corrected for urinary creatinine. Both patient groups had comparable levels of disease activity, as defined by the SLEDAI-2K, with no statistical difference between the two populations. In order to standardize histologic scoring a study specific scoring protocol was developed that contained all components required to complete ISN/RPS classification and calculate activity and chronicity indexes. A single renal pathologist, blinded to the biomarker data, scored the biopsies as per the protocol.

Linear modeling was used to refine the urinary proteins whose abundance differed significantly between disease states (all lupus patients vs control) and between types of disease (active renal vs active non-renal SLE). Forty-two urinary proteins (LN panel) showed significant differential abundance between active LN and active non-LN patients (FIG. 8 which is summarized as Table 1 herein). A number of unique proteins, not previously proposed as urinary LN biomarkers, as well as reported candidate LN biomarkers (e.g., adiponectin)¹² were identified. Several of these analytes effectively discriminate between active LN and non-LN patients (FIG. 2, Panel A), outperforming candidate biomarkers, such as urinary MCP-1, TWEAK and NGAL (FIG. 2, Panel B) that have been previously proposed in pediatric¹³⁻¹⁵ and adult^{16, 17} LN studies as activity-specific indicators. These results supported our hypothesis that a distinct urine protein pattern can discriminate between active LN and active non-LN patients. Through this unrestricted analysis we have identified a number of urinary proteins that identify patients with active LN (versus systemic disease) and that show promise as urinary biomarkers for monitoring disease activity in LN.

To be clinically useful in monitoring LN activity, a biomarker or panel of biomarkers must vary over time, reflecting changes in renal disease activity. To address this we examined the mutability of urinary adiponectin in a cohort of SLE patients (n=20) with active LN and with a prior history of LN with inactive disease (n=25) (FIG. 3). This analysis confirmed that urine adiponectin concentrations identified patients with active LN and that this analyte normalized with resolution of renal involvement. Therefore, as suggested by these results, it is expected that analytes of the LN panel reflect renal activity. Another requisite feature of LN-activity biomarkers is that they should reflect renal rather than systemic inflammation. The circulating plasma and urine concentration of adiponectin was determined in patients with active LN and a poor correlation was noted between these two measures, suggesting that increased levels in the urine reflected renal rather systemic activity (FIG. 3).

### Example 3

The Identification of Biomarkers that Predict Histopathologic Features of Lupus Nephritis.

As detailed above, patients with active LN were recruited at time of renal biopsy. To determine if protein levels correlated with histopathological variables (e.g., activity, chronicity) across patients, Spearman correlation followed by false discovery rate correction was performed. Ten proteins: Adiponectin, PAI-1, sgp130, IL-16, HGF, vWF, TIMP-1, Eotaxin, IP-10 and PDGF.BB (“biopsy panel”) were found to significantly correlate with activity score on renal biopsy (q<0.01). LN patients were then stratified based on histopathological score into (1) active proliferative lesions (ISN III or IV, (A or A/C)) or (2) non-proliferative or chronic lesions (ISN V, II or III (C), IV (C), VI). The capacity of individual components of the biopsy panel to discriminate between proliferative and non-proliferative/chronic renal lesions was examined with p values corrected for multiple testing. Five analytes (IP-10, vWF, adiponectin, IL-16 and PAI-1) strongly discriminated between proliferative and other biopsy proven renal lesions (FIG. 4). Notably, these analytes outperformed proteinuria (albumin/creatinine ratio) in the stratification of patients with active, proliferative renal lesions (FIG. 4). Based on this data it is proposed that these 5 analytes can be used generate a composite score to identify patient with proliferative renal lesions. The performance characteristics of these candidate biomarkers alone and in combination with clinical biomarkers (e.g. eGFR, proteinuria) is assessed for their ability to correctly classify renal proliferative lesions as described in Example 4. The identified candidate proliferative LN panel could serve to rule in the presence of active proliferative renal lesions avoiding unnecessary renal biopsies.

### Example 4

### Validation of Biomarker Panels

To identify potential novel urinary biomarkers, a proteomics approach was used to assess the levels of 128 urinary analytes in 60 active LN patients, 25 active non-LN patients (with no history of LN) and 24 healthy controls. Active LN was defined as the presence of one or more parameters of the renal SLEDAI-2K and/or confirmatory renal biopsy, whereas active non-LN was defined as a SLEDAI-2K of 7 (selected as >60% of physicians would alter treatment with scores of this magnitude)¹¹ with none of the renal parameters present. Both patient groups had comparable levels of disease activity, as defined by the SLEDAI-2K. Urinary analytes were measured by Luminex and results were corrected for urinary creatinine. The urinary proteins whose abundance differed significantly between disease states (SLE vs. healthy controls) and between study patients with and without LN were identified by performing linear modeling, with corrections for multiple testing. Notably, for several of the analytes the levels in active LN and non-LN patients were markedly different (FIG. 5A), outperforming candidate biomarkers, such as MCP-1, TWEAK and NGAL (FIG. 5B) that have been previously individually proposed as indicators of active LN.

To validate these biomarkers and to determine whether they discriminate between active LN and LN patients in remission, the levels of the urinary proteins defined in the discovery cohort were examined in an independent cohort of 33 SLE patients with active LN, 16 patients with active non-LN, and 30 LN patients in remission. Eighteen of the analytes (Adiponectin, PAI.1, IL.15, PF4.CXCL4, TIMP.1, albumin, vWF, sVCAM.1, IL.6, KIM-1, GRO, Cystatin C, CXCL6.GCP.2, IL.8, Clusterin, MMP.7, MCP.1, and GM.CSF) demonstrated significant differences between active LN and active non-LN in this smaller cohort (indicated by asterisks in Table 2, see also FIG. 6), with the majority (16/18, as given below) normalizing to levels similar to those observed in active non-LN patients in LN patients in remission. Notably, the levels of these proteins in active LN as compared to active non-LN patients were markedly different, outperforming previously published candidate biomarkers for active LN. Adiponectin, PAI.1, IL.15, PF4.CXCL4, TIMP.1, vWF, sVCAM.1, IL.6, KIM-1, GRO, Cystatin C, CXCL6.GCP.2, Clusterin, MMP.7, MCP.1, and GM.CSF were also significantly lower in LN patients that were in remission as compared to patients with active LN, indicating normalization with treatment. In addition, 4 urinary proteins were found to specifically be associated with active proliferative nephritis, as compared to non-proliferative/chronic nephritis, a discrimination which can currently only be made with an invasive renal biopsy. See FIGS. 6 and 7.

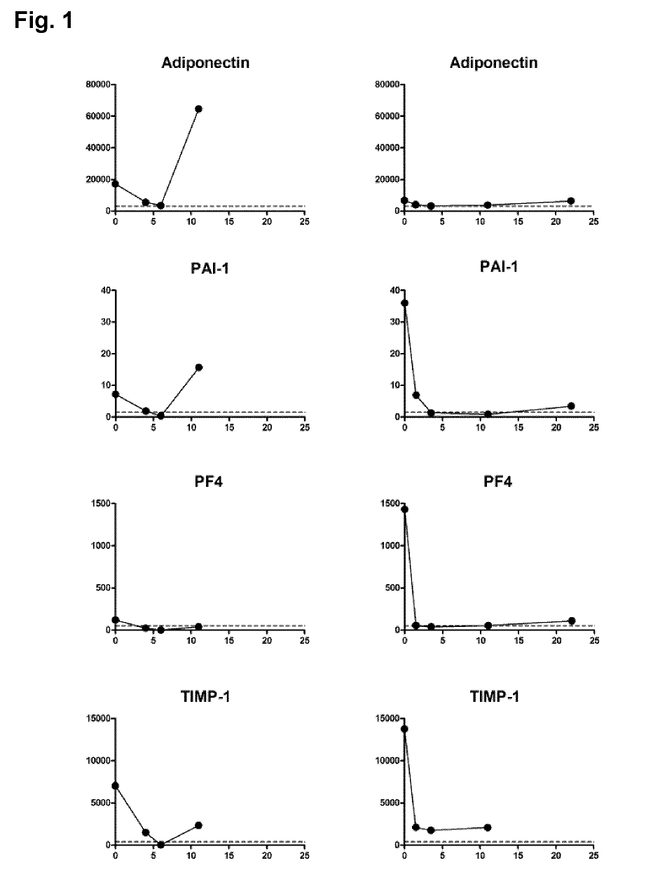

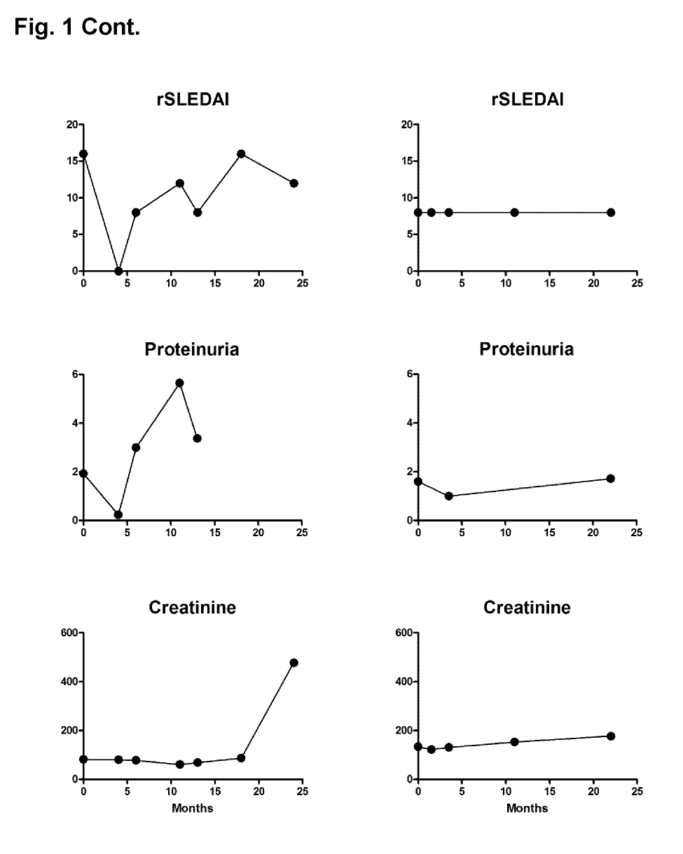

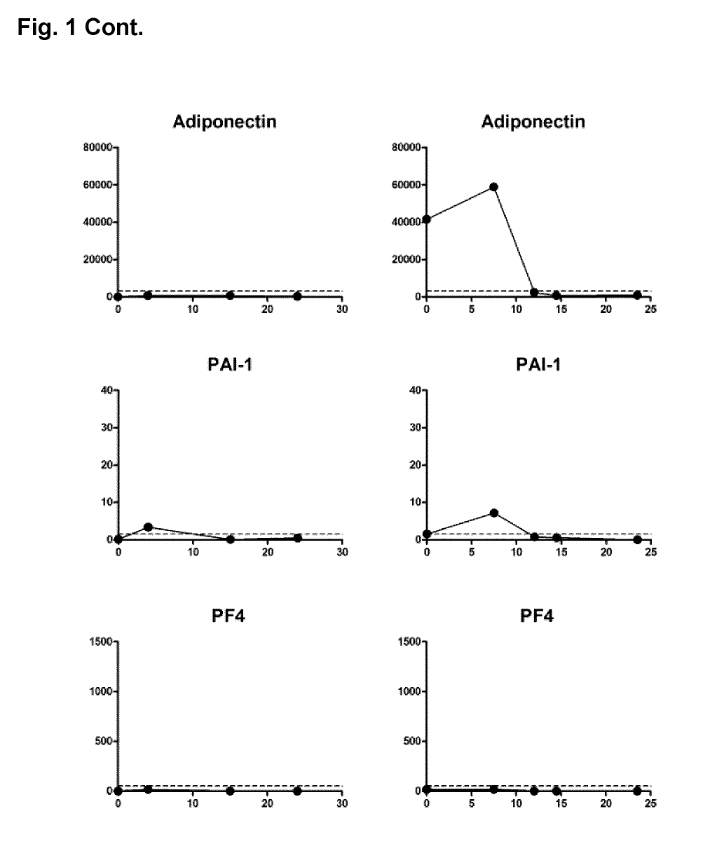

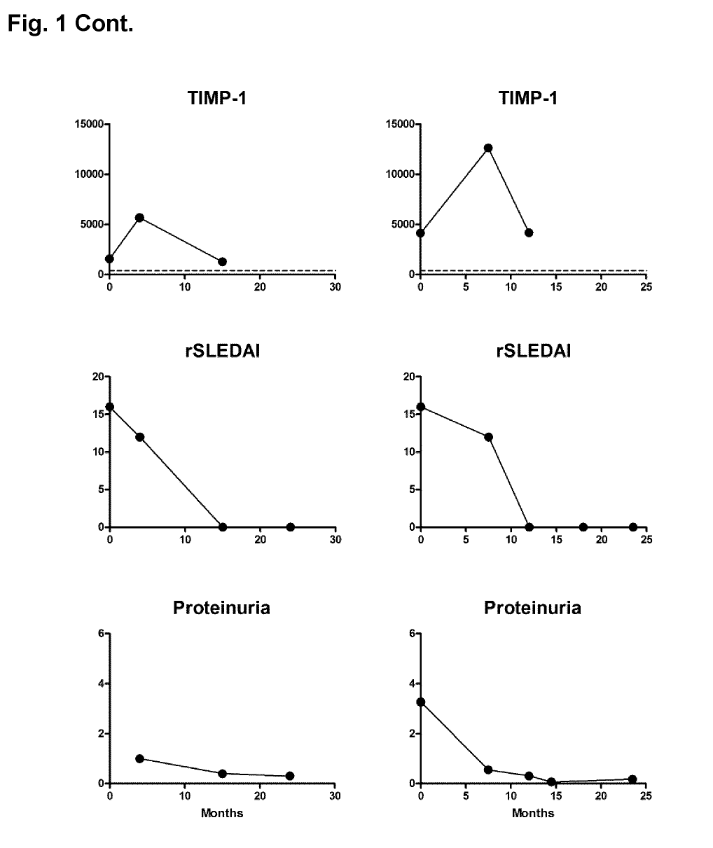

Preliminary results from serial measurements of active LN patients following treatment suggest that the failure to normalize these urinary analytes at 12-15 months, predicts treatment failure, supporting the potential clinical utility of repeated measures in SLE patients (see FIG. 1A, B). This Figure highlights two points. Firstly, renal inflammation, as indicated by elevated levels of urinary inflammatory markers, slowly resolves following treatment, normalizing in patients who achieve complete remission around 12-15 months. Secondly, the failure to normalize urinary inflammatory markers and/or an increase in these markers at 12 to 15 months is associated with treatment failure.

TABLE 1

| Urine | Fold | q |
|---|---|---|
| Analyte | Change* | value^{#} |
| MMP.2 | 2 | ≦10⁴ |
| Adiponectin | 4 | ≦10⁴ |
| PAI-1 | 4 | ≦10⁴ |
| Vwf | 3 | ≦10⁴ |
| MPO | 3 | ≦10⁴ |
| SVAM-1 | 3 | ≦10⁴ |
| Albumin | 3 | ≦10⁴ |
| IL.16 | 3 | ≦10⁴ |
| IL.15 | 3 | ≦10⁴ |
| IL.6 | 3 | ≦10⁴ |
| PF4 | 3 | 10³ |
| GRO | 2 | ≦10⁴ |
| MCP.1 | 2 | ≦10⁴ |
| SFas | 2 | ≦10⁴ |
| SCF | 2 | ≦10⁴ |
| HCF | 2 | ≦10⁴ |
| BCA.1 | 2 | ≦10⁴ |
| Clusterin | 2 | ≦10⁴ |
| Cystatin C | 2 | ≦10⁴ |
| CXCL7 | 2 | ≦10⁴ |
| KIM.1 | 2 | ≦10⁴ |
| SAP | 2 | 10³ |
| Eotaxin | 2 | 10² |
| MMP.7 | 2 | 10² |
| MMP.9 | 2 | 10² |
| IL.8 | 2 | 10² |
| TARC | 1 | ≦10⁴ |
| Perforin | 1 | ≦10⁴ |
| Eotaxin.2 | 1 | 10³ |
| TWEAK | 1 | 10³ |
| sgp30 | 1 | 10² |
| sIL-1R1 | 1 | 10² |
| TIMP.2 | 1 | 10² |
| GM.CSF | 1 | 10² |
| MCP.3 | 1 | 10² |
| VEGF | 1 | 10² |
| X6CKine | 3 (decrease) | ≦10⁴ |

*denotes Log₂ fold change rounded to nearest integer
^{#}denotes p value corrected for multiple testing

TABLE 2

| | | | | Padj |
|---|---|---|---|---|
| | Log₂ | | | Active |
| | Fold-change | | | LN vs |
| | Active LN vs | Padj | Padj | Remission |
| Urinary Analyte | NLN | Discovery | Replication | LN |
| Adiponectin* | 4 | ≦0.0001 | ≦0.0044 | ≦0.0018 |
| PAI-1* | 3.77 | ≦0.0001 | ≦0.0044 | ≦0.0018 |
| IL-15* | 3.32 | ≦0.0001 | ≦0.0044 | ≦0.0018 |
| PF4 (CXCL4)* | 3.04 | ≦0.0001 | ≦0.0044 | ≦0.0018 |
| TIMP-1* | 3.01 | ≦0.0001 | ≦0.0044 | ≦0.0018 |
| IL-16 | 2.97 | ≦0.0001 | 0.62 | N/A |
| Albumin* | 2.95 | ≦0.0001 | 0.022 | 1 |
| MPO | 2.62 | ≦0.0001 | 0.81 | N/A |
| vWF* | 2.5 | ≦0.0001 | ≦0.0044 | ≦0.0018 |
| sVCAM-1* | 2.49 | ≦0.0001 | ≦0.0044 | ≦0.0018 |
| IL-6* | 2.46 | ≦0.0001 | 0.0088 | ≦0.0018 |
| BCA-1 | 2.22 | ≦0.0001 | 0.54 | N/A |
| SAP | 2.19 | 0.001 | 0.33 | N/A |
| KIM-1* | 1.95 | ≦0.0001 | 0.044 | 0.013 |
| GRO* | 1.93 | ≦0.0001 | ≦0.0044 | 0.0054 |
| HCC-1 (CCL14a) | 1.91 | ≦0.0001 | ND | N/A |
| SCF | 1.89 | ≦0.0001 | 1 | N/A |
| MMP-9 | 1.89 | 0.007 | 1 | N/A |
| Cystatin C* | 1.87 | ≦0.0001 | 0.044 | 0.0072 |
| sFAS | 1.76 | ≦0.0001 | 0.18 | N/A |
| PDGF-BB | 1.7 | 0.005 | 0.80 | N/A |
| GCP-2 (CXCL6)* | 1.69 | ≦0.0001 | ≦0.0044 | ≦0.0018 |
| IL-8* | 1.66 | 0.002 | 0.0088 | 0.26 |
| Clusterin* | 1.58 | ≦0.0001 | ≦0.0044 | ≦0.0018 |
| Eotaxin | 1.58 | 0.002 | 1 | N/A |
| MMP-7* | 1.56 | 0.002 | 0.022 | ≦0.0018 |
| MMP-2 | 1.56 | ≦0.0001 | 1 | N/A |
| TARC | 1.48 | ≦0.0001 | 1 | N/A |
| HGF | 1.47 | ≦0.0001 | 0.44 | N/A |
| NAP-2 (CXCL7) | 1.47 | 0.001 | 1 | N/A |
| Perforin | 1.37 | ≦0.0001 | 1 | N/A |
| MCP-1* | 1.34 | ≦0.0001 | ≦0.0044 | ≦0.0018 |
| IFN-gamma | 1.29 | 0.005 | 1 | N/A |
| GM-CSF* | 1.24 | 0.007 | 0.0088 | 0.013 |
| Eotaxin-2 | 1.22 | ≦0.0001 | 1 | N/A |
| TWEAK | 1.16 | 0.001 | 0.54 | N/A |
| I-TAC (CXCL11) | 1.13 | 0.007 | 0.079 | N/A |
| IL-1RI | 1.12 | 0.003 | 1 | N/A |
| Sgp130 | 1.07 | ≦0.0001 | 1 | N/A |
| sTNFRI | 1.06 | 0.004 | 1 | N/A |
| MCP-3 | 1.02 | 0.006 | 1 | N/A |
| TIMP-2 | 1.01 | 0.002 | 1 | N/A |
| NGAL | .95 | 0.01 | ND | N/A |
| VEGF | .72 | 0.007 | 1 | N/A |
| X6CKine | −3.15 | ≦0.0001 | 1 | N/A |

*denotes analytes that remained statistically significant when comparing active LN and active non-LN in the validation cohort.

### CITATIONS FOR REFERENCES REFERRED TO IN THE SPECIFICATION

1. Barsalou J, Levy D M, Silverman E D. An update on childhood-onset systemic lupus erythematosus. Current opinion in rheumatology 2013; 25:616-22.
2. Mina R, Brunner H I. Update on differences between childhood-onset and adult-onset systemic lupus erythematosus. Arthritis Res Ther 2013; 15:218.
3. Weening J J, D'Agati V D, Schwartz M M, et al. The classification of glomerulonephritis in systemic lupus erythematosus revisited. J Am Soc Nephrol 2004; 15:241-50.
4. Rovin B H, Birmingham D J, Nagaraja H N, Yu C Y, Hebert L A. Biomarker discovery in human SLE nephritis. Bull NYU Hosp Jt Dis 2007; 65:187-93.
5. El Hachmi M, Jadoul M, Lefebvre C, Depresseux G, Houssiau F A. Relapses of lupus nephritis: incidence, risk factors, serology and impact on outcome. Lupus 2003; 12:692-6.
6. Touma Z, Urowitz M B, Ibanez D, Gladman D D. Time to recovery from proteinuria in patients with lupus nephritis receiving standard treatment. The Journal of rheumatology 2014; 41:688-97.
7. El-Zoghby Z M, Grande J P, Fraile M G, Norby S M, Fervenza F C, Cosio F G. Recurrent idiopathic membranous nephropathy: early diagnosis by protocol biopsies and treatment with anti-CD20 monoclonal antibodies. American journal of transplantation: official journal of the American Society of Transplantation and the American Society of Transplant Surgeons 2009; 9:2800-7.
8. Beck L H, Jr., Fervenza F C, Beck D M, et al. Rituximab-induced depletion of anti-PLA2R autoantibodies predicts response in membranous nephropathy. J Am Soc Nephrol 2011; 22:1543-50.
9. Beck L H, Jr., Salant D J. Membranous nephropathy: recent travels and new roads ahead. Kidney international 2010; 77:765-70.
10. Gladman D D, Ibanez D, Urowitz M B. Systemic lupus erythematosus disease activity index 2000. The Journal of rheumatology 2002; 29:288-91.
11. Abrahamowicz M, Fortin P R, du Berger R, Nayak V, Neville C, Liang M H. The relationship between disease activity and expert physician's decision to start major treatment in active systemic lupus erythematosus: a decision aid for development of entry criteria for clinical trials. The Journal of rheumatology 1998; 25:277-84.
12. Rovin B H, Song H, Hebert L A, et al. Plasma, urine, and renal expression of adiponectin in human systemic lupus erythematosus. Kidney international 2005; 68:1825-33.
13. Watson L, Midgley A, Pilkington C, et al. Urinary monocyte chemoattractant protein 1 and alpha 1 acid glycoprotein as biomarkers of renal disease activity in juvenile-onset systemic lupus erythematosus. Lupus 2012; 21:496-501.
14. Hinze C H, Suzuki M, Klein-Gitelman M, et al. Neutrophil gelatinase-associated lipocalin is a predictor of the course of global and renal childhood-onset systemic lupus erythematosus disease activity. Arthritis Rheum 2009; 60:2772-81.
15. Brunner H I, Mueller M, Rutherford C, et al. Urinary neutrophil gelatinase-associated lipocalin as a biomarker of nephritis in childhood-onset systemic lupus erythematosus. Arthritis Rheum 2006; 54:2577-84.
16. Abujam B, Cheekatla S, Aggarwal A. Urinary CXCL-10/IP-10 and MCP-1 as markers to assess activity of lupus nephritis. Lupus 2013; 22:614-23.
17. Schwartz N, Rubinstein T, Burkly L C, et al. Urinary TWEAK as a biomarker of lupus nephritis: a multicenter cohort study. Arthritis Res Ther 2009; 11:R143.
18. Rovin B H, Furie R, Latinis K, et al. Efficacy and safety of rituximab in patients with active proliferative lupus nephritis: the Lupus Nephritis Assessment with Rituximab study. Arthritis Rheum 2012; 64:1215-26.
19. Schwartz G J, Munoz A, Schneider M F, et al. New equations to estimate GFR in children with CKD. J Am Soc Nephrol 2009; 20:629-37.
20. Rovin B H, Song H, Birmingham D J, Hebert L A, Yu C Y, Nagaraja H N. Urine chemokines as biomarkers of human systemic lupus erythematosus activity. J Am Soc Nephrol 2005; 16:467-73.
21. Tian S, Li J, Wang L, et al. Urinary levels of RANTES and M-CSF are predictors of lupus nephritis flare. Inflammation research: official journal of the European Histamine Research Society [et al] 2007; 56:304-10.

1. An SLE and/or lupus nephritis (LN) biomarker panel comprising a solid support and two or more biomarker detection agents each biomarker detection agent specific for a corresponding target biomarker selected from a group of target biomarkers consisting of PAI-1, vWF, Adiponectin, CCL20.MIP.3A, CXCL6.GCP.2, CXCL11.I.TAC, CCL14a.HCC.1, CCL19.MIP.3B, sTNFRI, TIMP.1, IFN-gamma, beta-2-microglobulin, CXCL7.NAP.2, sVCAM.1, TWEAK, sgp130, sIL.1R1, KIM.1, albumin, clusterin, cystatin C, eotaxin.2, BCA.1, IL.16, TARC, X6CKine, SCF, HGF, SAP, PF4.CXCL4, myeloperoxidase, sFas, perforin, MMP.2, MMP.7, MMP.9, TIMP.2, eotaxin, GM.CSF, GRO, MCP.3, IL.15, IL.6, IL.8, MCP.1, VEGF, NOI, SVAM-1, MCP.1, HCF, CXCL7, sgp30, IP.10, PDGF-BB, and NGAL.

2. The biomarker panel wherein each biomarker detection agent is an antibody or binding fragment thereof.

3. (canceled)

4. The biomarker panel of claim 1 wherein the target biomarker is selected from CXCL6.GCP.2, TIMP.1, Adiponectin, PAI.1, sVCAM.1, sgp130, Albumin, Clusterin, Cystatin C, IL.16, HGF, PF4.CXCL4, vWF, MMP.7, Eotaxin, GM.CSF, GRO, IL.15, IL.6, MCP.1, IP-10, and PDGF-BB.

5. (canceled)

6. (canceled)

7. The biomarker panel of claim 1 herein there are at least three of the two or more biomarker detection agents and the corresponding target biomarkers comprise at least three target biomarkers selected from TIMP.1, Adiponectin, PAI.1, sVCAM.1, vWF, PF4.CXCL4, IL.15, and NGAL.

8. The biomarker panel of claim 1 wherein the solid support is a bead, a well plate, or a chip.

9. The biomarker panel of claim 8 wherein the bead comprises a unique code optionally a colour code and each unique code is associated with each biomarker detection agent.

10. A kit comprising the biomarker panel of claim 1 and one or more of:
i. sample dilution buffer;
ii. wash buffer;
iii. filter;
iv. positive control; and/or
v. instructions for performing a method described herein.

11. A method of measuring a level using one or more target biomarkers in a urine sample comprising contacting a urine sample with the biomarker panel of claim 1 under conditions for forming a complex between the one or more target biomarkers in the urine sample and one or more of the two or more biomarker detection agents; and quantifying the amount of complex formed for one or more of the target biomarkers.

12. The method of claim 11, wherein the urine sample is obtained from a subject with Systemic Lupus Erythematosus (SLE) or suspected of having SLE.

13. The method of claim 12, wherein the method is for diagnosing SLE and an increase in one or more target biomarker levels relative to a control and/or a decrease in one of the target biomarker levels relative to a control indicates the subject has SLE.

14. The method of claim 12, wherein the method is for monitoring disease activity.

15. The method of claim 12, wherein the method is for distinguishing active SLE with LN from active SLE without LN.

16. The method of claim 12, wherein the urine sample is obtained from a subject that has received or is receiving treatment for LN and a decrease in one or more target biomarker levels shown to be increased in the LN biomarker set, reduced biomarker set, or screening biomarker set as compared to the control and/or an increase in one or more of the target biomarker levels shown to be decreased in the LN biomarker set, reduced biomarker set, or screening biomarker set as compared to the control indicates the subject has responded or is responding to the treatment.

17. (canceled)

18. The method of claim 12, wherein an increased level compared to a control in one or more of the target biomarkers is indicative of active proliferative renal lesions.

19. The method of claim 12, wherein the method further comprises administering an active proliferative lesion suitable treatment if an increased level of one or more of the biomarkers is detected and a non-proliferative/chronic lesion suitable treatment if a lack of increased level of one or more of the biomarkers is detected.

20. The method of claim 11, wherein 2, 3, 4, 5, 6, 7, 8, 9, 10, 11, 12, 13,14, 15, 16, 17, 18, 19, 20, 21, 22, 23, 24, 25, 26, 27, 28, 29, 30, 31, 32, 33, 34, 35, 36 and/or 37 target biomarker levels are measured.

21. (canceled)

22. The method of claim 11, wherein the biomarker level is a standardized level, optionally standardized to creatinine.

23. The method of claim 11, wherein one or more SLE or LN clinical markers are also assessed.

24. The method of claim 11, wherein biomarker level is measured using a multiplex assay system.

25. (canceled)

26. The method of claim 13, in which there are at least three biomarker levels measured and the diagnosis is positive when at least two of the three are increased by at least 2 fold.

27. (canceled)
